(19) United States
(12) Patent Application Publication
(10) Pub. No.: US 2022/0194977 A1
(43) Pub. Date: Jun. 23, 2022

(54) NEW PROCESS FOR PREPARING SUGAMMADEX

(71) Applicant: PROCOS S.P.A., Cameri (IT)

(72) Inventors: Fabio Morana, Novara (IT); Jacopo Roletto, Torino (IT); Paolo Paissoni, Druento (IT)

(73) Assignee: PROCOS S.P.A., Cameri (IT)

(21) Appl. No.: 17/594,455

(22) PCT Filed: Apr. 23, 2020
(86) PCT No.: PCT/IB20/53857

§ 371 Date: Oct. 17, 2021

(30) Foreign Application Priority Data
Apr. 24, 2019 (IT) 102019000006328

(51) Int. Cl. C07H 15/14

(52) U.S. Cl. CPC C07H 15/14

(57) ABSTRACT

The present invention relates to a new process for preparing Sugammadex.

### FIELD OF THE INVENTION

The present invention relates to a new process for preparing Sugammadex.

### STATE OF THE ART

Sugammadex is a selective muscle relaxant antagonist capable of cancelling out the action, for example, of rocuronium and vecuronium, and is marketed under the name Bridion® in the form of a sterile solution for intravenous injection.

Sugammadex is the 6-per-deoxy-6-per(2-carboxyethyl)thio-γ-cyclodextrin octasodium salt, represented by the following structural formula:

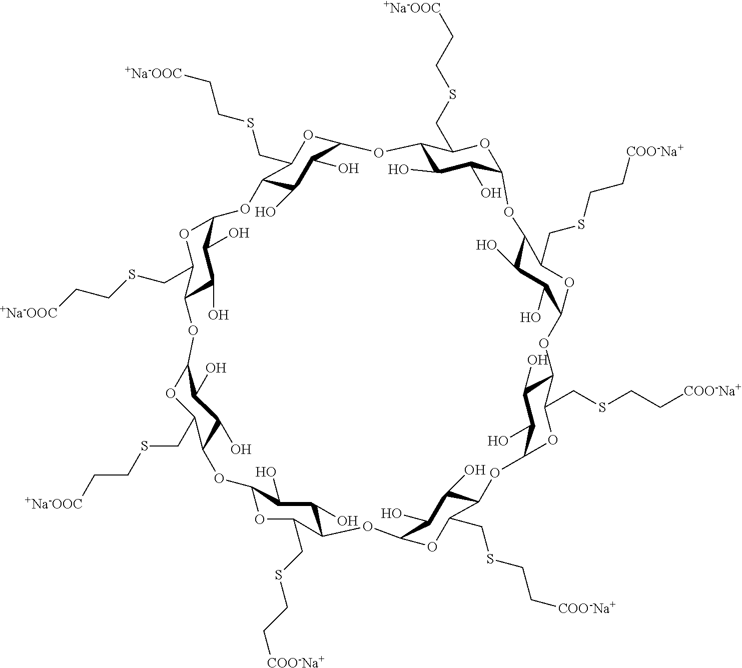

Different synthesis methods of Sugammadex are known in the literature involving an intermediate compound of formula (I):

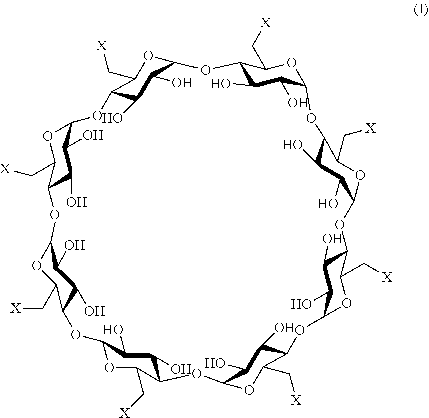

wherein X is a halogen substituent, which is then reacted, by means of a substitution reaction into an aprotic organic solvent, with 2-mercaptopropionic acid or a derivative thereof, to give Sugammadex.

The Applicant has found that the synthesis methods of Sugammadex involving the compound of formula (I) have technological limits related to the difficulty of reaching high yield values and to the accumulation of partial substitution products, such as in particular the compound of formula (II):

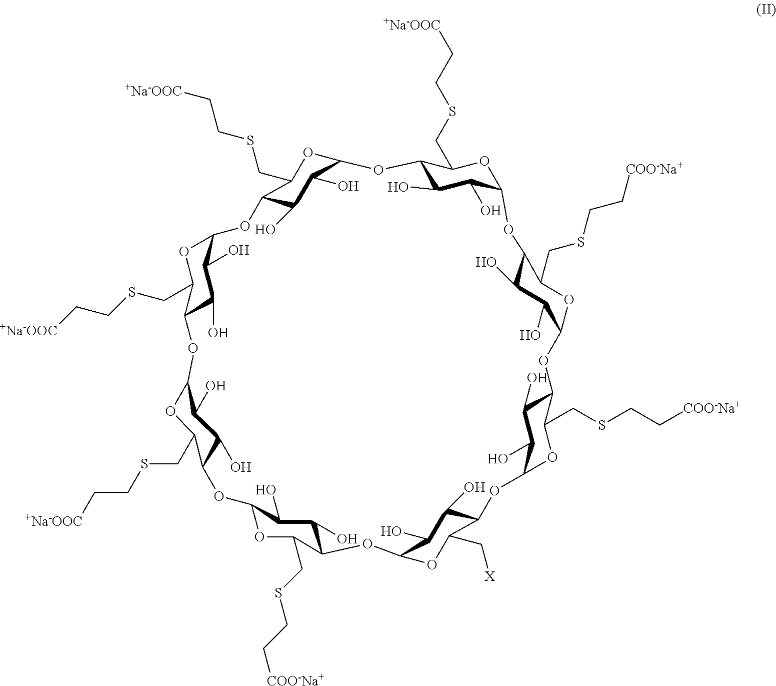

wherein X is a halogen substituent. The Applicant has also found that the removal of these partial substitution products is particularly difficult to implement due to the high structural similarity of the same to Sugammadex, even by using multiple passages of isolation of the product from the reaction medium and subsequent purification steps thereof, unless complex and expensive purification techniques such as chromatographic methods, molecular exclusion membranes and ion exchange resins are used. The Applicant has also found that complex and expensive purification processes can constitute a limit to the competitiveness of the synthesis process of Sugammadex, in terms of time and costs.

The Applicant has also found that the presence of partial substitution products having high structural similarity to Sugammadex also hinders the achievement of sufficiently high purity values for Sugammadex itself. The Applicant has noted that this is a critical aspect of fundamental importance, considering that, for example, the guidelines “Impurities in new drug substances” (Q3A) of the International Council for Harmonization of Technical Requirements for Pharmaceuticals for Human Use (ICH) prescribe a maximum content of tolerable impurities for the registration of active ingredients in the pharmaceutical field. In particular, the Applicant has found that the aforesaid ICH guidelines identify the limits for known impurities at 0.1% and for unknown impurities at 0.10%.

### SUMMARY OF THE INVENTION

The aim of the present invention is therefore to provide a new and competitive process for the synthesis of Sugammadex capable of reaching high yield values and at the same time ensuring the formation of lesser amounts of partial substitution products that are difficult to separate from Sugammadex, among which in particular the compound of formula (II):

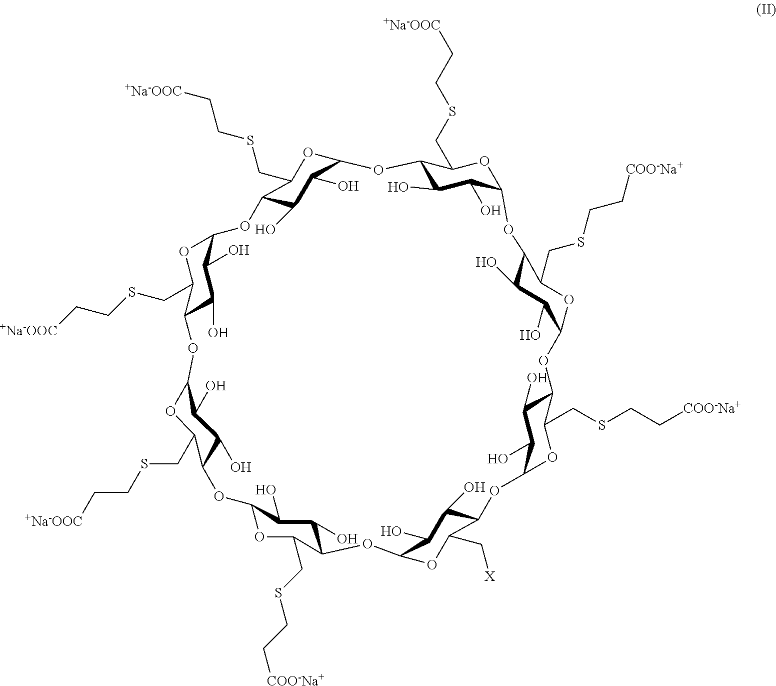

wherein X is a halogen substituent, so as to allow easier, effective and economic isolation and purification of Sugammadex from the reaction mixture.

In accordance with the present invention, the Applicant has surprisingly found that it is possible to achieve the aforesaid aim by using particular reaction conditions and technical measures during the synthesis step which provides for the substitution reaction of said intermediate compound of formula (I) with 2-mercaptopropionic acid or a derivative thereof in an aprotic organic solvent, to give Sugammadex.

In particular, the Applicant has discovered that, by adding a specific and defined amount of water into the reaction mixture in a specific stage of progress of said substitution reaction, it is possible to improve the reaction yield and at the same time ensure the presence, at the end of the reaction itself, of lesser amounts of partial substitution products that are difficult to separate from the product, including in particular the compound of formula (II). Moreover, this additionally makes the subsequent Sugammadex purification step simpler and more competitive.

Therefore, the present invention relates in a first aspect thereof to a process for preparing the 6-per-deoxy-6-per(2-carboxyethyl)thio-γ-cyclodextrin octasodium salt, comprising the steps of:

a. reacting a compound of formula (I)

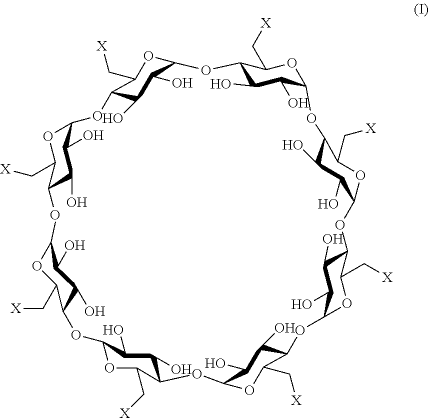

wherein X is selected from the group consisting of: CI, and Br, with 3-mercaptopropionic acid in presence of at least one sodium alkoxide and of at least one aprotic organic solvent;

b. adding to the reaction mixture of step a. water, in an amount of from 0.5% to 10% by volume with respect to the total volume of said at least one aprotic organic solvent, when the compound of formula (II)

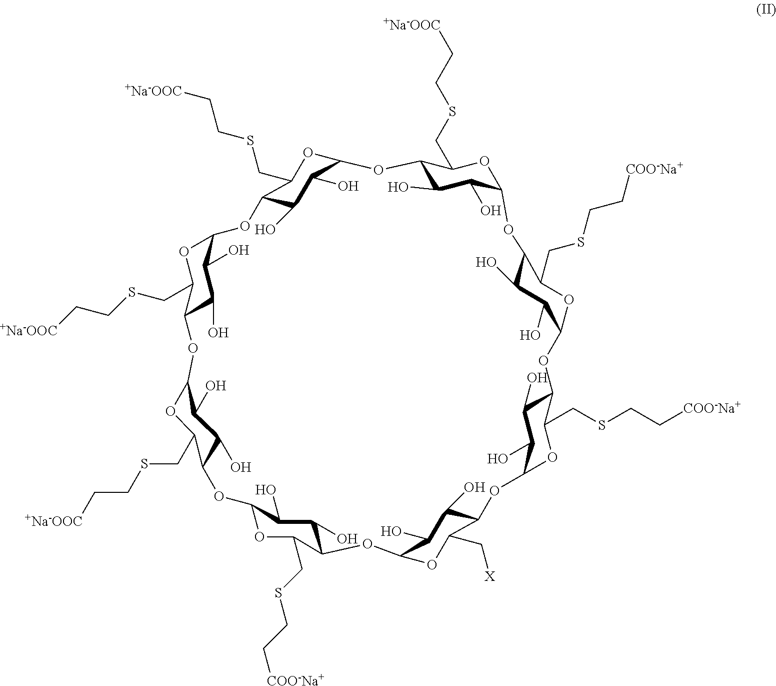

wherein X as defined above is present in the reaction mixture in an amount equal to or lower than 10% with respect to the total mass of reaction; and

c. isolating the 6-per-deoxy-6-per(2-carboxyethyl)thio-γ-cyclodextrin octasodium salt from the total mass of reaction obtained from step b.

In fact, it has surprisingly been discovered that, thanks to the presence of a specific and defined amount of water in the reaction mixture at a specific stage of the substitution reaction identified with a “threshold” value of the degree of progress of the reaction, it is possible to improve the reaction yield and at the same time ensure the presence, at the end of the reaction, of lesser amounts of partial substitution products that are difficult to separate from the product.

The Applicant has in fact observed that as the substitution reaction progresses, a precipitate gradually forms, in which the Applicant believes that the partial substitution products are incorporated, thus being subtracted from the substitution reaction, which therefore no longer progresses beyond a certain limit. Without thereby willing to be bound to a specific theory, the Applicant believes that the addition of a specific and defined amount of water into the reaction mixture when the amount of the compound of formula (II) is equal to or lower than 10% with respect to the total mass of reaction, allows reducing the amount of precipitate and thus limiting the incorporation of partial substitution products which are therefore no longer subtracted from the substitution reaction. In this way, the Applicant has unexpectedly identified the possibility of improving the reaction yield and at the same time ensuring the presence, at the end of the reaction, of lesser amounts of partial substitution products that are difficult to separate from the product.

The Applicant has also noted that the synthesis processes of Sugammadex involving said compound of formula (I), also have technological limits relating to the formation of unwanted amounts of gaseous by-products during the reaction due to the in situ synthesis of the halogenating agent. Said gaseous by-products in their evolution can, on the one hand, give rise to phenomena of entrainment of the reaction product which limit the reaction yield and, on the other, lead to accumulations in the equipment, for example in the condensers, thus reducing the efficiency of the equipment itself.

In a second aspect, the present invention also relates to an improved process for preparing a compound of formula (I)

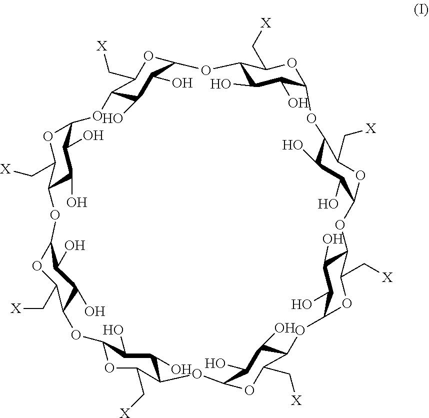

wherein X is selected from the group consisting of: CI, and Br, comprising the steps of:
A) reacting, in presence of at least one solvent selected from the group consisting of: toluene, and dichloromethane, at least one halide selected from the group consisting of: oxalyl halide, and thionyl halide, and a compound of formula (IV)

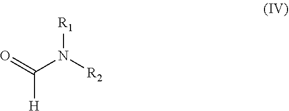

wherein R₁ and R₂ are —CH₃, phenyl groups or represent together a —CH₂—CH₂—O—CH₂—CH₂-group, thus obtaining a compound of formula (III)

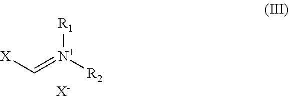

wherein

R₁, R₂ and X are as defined above;
B) distilling from the reaction mixture of step A) the at least one solvent selected from the group consisting of: toluene, and dichloromethane; and
C) reacting in presence of dimethylformamide at least one γ-cyclodextrin with the compound of formula (III) obtained from step B), thus obtaining the compound of formula (I).

In fact, the Applicant has found that the present process for preparing the compound of formula (I) allows obtaining high yields of the desired product under safe conditions and avoiding the formation of undesired amounts of gaseous by-products and therefore without giving rise to phenomena of entrainment of the reaction product or accumulations in the equipment.

The process for preparing the compound of formula (I) according to the present invention can be applied in any synthesis process of Sugammadex described in the prior art which provides for the involvement of the compound of formula (I) itself.

Advantageously, the Applicant has further found that the process for preparing the compound of formula (I) according to the second aspect of the present invention can be applied upstream of the process for preparing the 6-per-deoxy-6-per(2-carboxyethyl)thio-γ-cyclodextrin octasodium salt according to the first aspect of the present invention.

In a third and preferred aspect, therefore, the present invention relates to a process for preparing the 6-per-deoxy-6-per(2-carboxyethyl)thio-γ-cyclodextrin octasodium salt, comprising the steps of:

(i) preparing a compound of formula (I) by means of the process according to the second aspect of the present invention;

(ii) preparing the 6-per-deoxy-6-per(2-carboxyethyl)thio-γ-cyclodextrin octasodium salt by means of the process according to the first aspect of the present invention.

### BRIEF DESCRIPTION OF THE DRAWINGS

FIG. 1 shows the X-ray diffractogram of the amorphous 6-per-deoxy-6-per-chloro-γcyclodextrin obtained in Example 1.

FIG. 2 shows the mass spectrum of the compound of formula (II) of Example 9;

### DETAILED DESCRIPTION OF THE INVENTION

The present invention relates, in a first aspect thereof, to a process for preparing the 6-per-deoxy-6-per(2-carboxyethyl)thio-γ-cyclodextrin octasodium salt, comprising the steps of:

a. reacting a compound of formula (I)

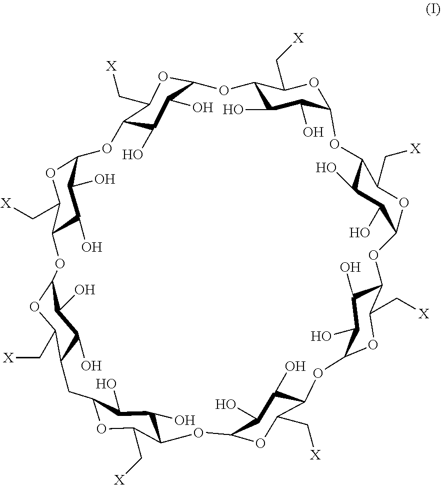

wherein X is selected from the group consisting of: Cl, and Br, with 3-mercaptopropionic acid in presence of at least one sodium alkoxide and of at least one aprotic organic solvent;

b. adding to the reaction mixture of step a. water, in an amount of from 0.5% to 10% by volume with respect to the total volume of said at least one aprotic organic solvent, when the compound of formula (II)

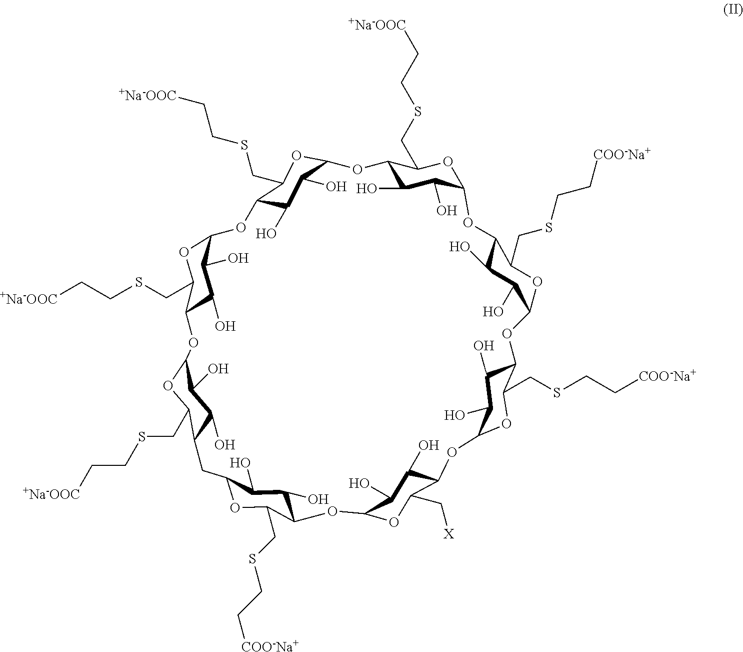

wherein X is as defined above, is present in the reaction mixture in an amount equal to or lower than 10% with respect to the total mass of reaction; and

c. isolating the 6-per-deoxy-6-per(2-carboxyethyl)thio-γ-cyclodextrin octasodium salt from the total mass of reaction obtained from step b.

The Applicant has in fact discovered that, by adding a specific and defined amount of water in the reaction mixture of step b., it is possible to improve the reaction yield and at the same time ensure the presence, at the end of the reaction, of lesser amounts of partial substitution products that are difficult to separate from the product, including in particular the compound of formula (II).

The present invention can have, in one or more aspects thereof, one or more of the preferred characteristics set forth below, which can be combined as desired with each other according to the application requirements.

Within the context of the present description and following claims, all the numerical magnitudes indicating amounts, parameters, percentages, and so on are to be considered preceded in every circumstance by the term “about” unless indicated otherwise. Further, all the ranges of numerical magnitudes include all the possible combinations of maximum and minimum numerical values and all the possible intermediate ranges, in addition to those indicated below.

In the present invention, when the expression “total mass of reaction” is used, it refers to the set made up of the compound of formula (I), of the 6-per-deoxy-6-per(2-carboxyethyl)thio-γ-cyclodextrin octasodium salt, of the compound of formula (II) and of all the other partial substitution products and by-products that are formed during the process according to the present invention.

The process for preparing Sugammadex involves step a. reacting a compound of formula (I) with 3-mercaptopropionic acid in presence of at least one sodium alkoxide and at least one aprotic organic solvent.

One among the advantages of the process according to the present invention is that the form in which the compound of formula (I) is used in step a. is in no way limited, thereby helping to make the process extremely flexible and adaptable. For example, the compound of formula (I) can be advantageously used in the process according to the present invention in an amorphous form, where the amorphous nature of the compound can be verified by routine analyses, such as for example X-ray diffractometry, and can, for example, be supplied directly from the market.

Examples of commercially available compounds of formula (I) are, for example, those marketed by Apollo Scientific ltd (United Kingdom), by Carbosynth Limited (United Kingdom), by Sagechem Limited (China), Toronto Research Chemicals (Canada) under the names “6-chloro-6-deoxy-gamma-cyclodextrin” or “octakis(6-deoxy-6-chlorine)-gamma-cyclodextrin”.

In an embodiment of the present invention, the compound of formula (I) is prepared before or during said step a. by means of a process comprising reacting at least one γ-cyclodextrin with at least one halogenating agent in presence of dimethylformamide. Preferably, when the compound of formula (I) is prepared before said step a., the compound of formula (I) is isolated at the end of the reaction of the at least one γ-cyclodextrin with the at least one halogenating agent.

Preferably, said reaction of the at least one γ-cyclodextrin with the at least one halogenating agent in presence of dimethylformamide is carried out at a temperature in the range from 40 to 70° C.

Preferably, the at least one halogenating agent is used in amounts from 8 to 50 equivalents with respect to the equivalents of the at least one γ-cyclodextrin, more preferably from 12 to 40 equivalents with respect to the equivalents of the at least one γ-cyclodextrin, even more preferably from 20 to 35 equivalents with respect to the equivalents of the at least one γ-cyclodextrin.

The at least one halogenating agent can be selected from any of the halogenating agents known for this purpose to the skilled person in the art.

Preferably, the at least one halogenating agent is a compound of formula (III)

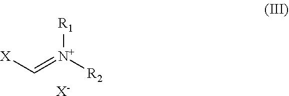

wherein

R₁ and R₂ are —CH₃, phenyl groups or represent together a-CH₂—CH₂—O—CH₂—CH₂-group; and

X is selected from the group consisting of: CI, and Br.

Said compound of formula (III) can be formed in situ, or can be obtained, and optionally isolated, before reacting the at least one γ-cyclodextrin with the at least one halogenating agent in presence of dimethylformamide.

In a preferred embodiment, said compound of formula (III) is obtained by reacting at least one compound selected from the group consisting of: dimethylformamide, N-formyl morpholine, and diphenylformamide, with at least one halide selected from the group consisting of: oxalyl halide, and thionyl halide.

In a further preferred embodiment, said compound of formula (III) is obtained before reacting the at least one γ-cyclodextrin with the at least one halogenating agent in presence of dimethylformamide, by means of the steps of:
reacting, in presence of at least one solvent selected from the group consisting of: toluene, and dichloromethane, at least one halide selected from the group consisting of: oxalyl halide, and thionyl halide, and a compound of formula (IV)

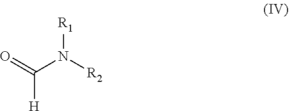

wherein R₁ and R₂ are —CH₃, phenyl groups or represent together a —CH₂—CH₂—O—CH₂—CH₂-group; and
distilling the at least one solvent selected from the group consisting of: toluene, and dichloromethane.

In said preferred embodiment, the at least one halide and said compound of formula (IV) can be used in different reciprocal ratios.

In a preferred embodiment, the at least one halide is used in excess with respect to the equivalents of the compound of formula (IV), more preferably in an amount from 1.00 to 2.00 equivalents with respect to the equivalents of the compound of formula (IV), even more preferably in an amount from 1.05 to 1.9 equivalents with respect to the equivalents of the compound of formula (IV), even more preferably in an amount from 1.10 to 1.8 equivalents with respect to the equivalents of the compound of formula (IV). Preferably, in said embodiment, the excess halide is removed at the end of the reaction with the compound of formula (IV).

In a further particularly preferred embodiment, the at least one halide is used in defect with respect to the equivalents of the compound of formula (IV), more preferably in an amount from 0.10 to 0.95 equivalents with respect to the equivalents of the compound of formula (IV), even more preferably in an amount from 0.40 to 0.80 equivalents with respect to the equivalents of the compound of formula (IV).

Preferably, in step a. of the process according to the present invention the at least one sodium alkoxide is selected from the group consisting of sodium tert-butoxide, sodium methoxide, sodium ethoxide, and sodium tert-pentoxide.

Preferably, in step a. of the process according to the present invention, the at least one aprotic organic solvent is dimethylsulfoxide or a mixture of solvents comprising dimethylsulfoxide and at least another solvent selected from the group consisting of: tetrahydrofuran, dimethylformamide, dimethylacetamide, N-methylpyrrolidone, methanol, ethanol, n-propanol, isopropanol, n-butanol, isobutanol, and ethylene glycol.

Preferably, said mixture of solvents is a binary mixture consisting of dimethylsulfoxide and at least one further solvent selected from the group consisting of: tetrahydrofuran, dimethylformamide, dimethylacetamide, N-methylpyrrolidone, methanol, ethanol, n-propanol, isopropanol, n-butanol, isobutanol, and ethylene glycol.

Preferably, said mixture of solvents is a binary mixture consisting of dimethylsulfoxide and at least one further solvent selected from the group consisting of: tetrahydrofuran, dimethylformamide, dimethylacetamide, N-methylpyrrolidone, wherein dimethylsulfoxide is present in an amount from 16% to 96% by volume, more preferably from 54% to 87% by volume.

According to a further preferred embodiment, said mixture of solvents is a binary mixture consisting of dimethylsulfoxide and at least one further solvent selected from the group consisting of: methanol, ethanol, n-propanol, isopropanol, n-butanol, isobutanol, and ethylene glycol, wherein dimethylsulfoxide is present in an amount from 80% to 99% by volume, preferably from 85% to 99% by volume, more preferably from 90% to 99% by volume, even more preferably from 90% to 98% by volume. Preferably, in said further preferred embodiment said further solvent is advantageously selected from the group consisting of: methanol, ethanol, n-propanol, isopropanol.

Preferably, said mixture of solvents is a ternary mixture comprising dimethylsulfoxide, tetrahydrofuran and a third solvent selected from the group consisting of: dimethylformamide, dimethylacetamide, and N-methylpyrrolidone.

In a preferred embodiment, in said ternary mixture the volume ratio of dimethylsulfoxide:tetrahydrofuran:third solvent ranges from 25:1:1 to 0.2:1:1.

Preferably, step a. of the process according to the present invention is carried out at a temperature from 40 to 70° C.

Preferably, in step a. of the process according to the present invention, the at least one sodium alkoxide is used in an amount from 16 to 40 equivalents with respect to the equivalents of the compound of formula (I), more preferably in an amount from 20 to 30 equivalents with respect to the equivalents of the compound of formula (I).

Preferably, in said step a. the 3-mercaptopropionic acid is used in an amount from 8 to 20 equivalents with respect to the equivalents of the compound of formula (I), more preferably in an amount from 10 to 15 equivalents with respect to the equivalents of the compound of formula (I).

The process according to the present invention comprises step b. adding to the reaction mixture of step a. water, in an amount of from 0.5% to 10% by volume with respect to the total volume of said at least one aprotic organic solvent, when the compound of formula (II) is present in the reaction mixture in an amount equal to or lower than 10% with respect to the total mass of reaction, even more preferably in an amount equal to or lower than 5% with respect to the total mass of reaction. The amount of compound of formula (II) in the total mass of reaction can be easily monitored by means of any analysis technique on samples of the reaction mixture (such as for example high pressure liquid chromatography, HPLC).

Preferably, the amount of compound of formula (II) in the total mass of reaction is measured by means of high pressure liquid chromatography, (HPLC) determining the percentage value of the chromatographic area of the compound of formula (II) with respect to the value of the chromatographic area of the total mass of reaction.

Even more preferably, said determination of the percentage of the chromatographic area of the compound of formula (II) with respect to the value of the chromatographic area of the total mass of reaction is carried out by means of a UV-visible spectrophotometric diode array detector (UV-DAD) set at a wavelength of 210 nm.

Preferably in said step b. of the process according to the present invention, water is added in an amount of from 1% to 5% by volume with respect to the total volume of said at least one aprotic organic solvent.

Preferably, step b. of the process according to the present invention is carried out at a temperature from 40 to 70 ° C.

The process according to the present invention comprises step c. isolating the 6-per-deoxy-6-per(2-carboxyethyl)thio-γ-cyclodextrin octasodium salt from the total mass of reaction obtained from step b.

Preferably, in said step c. the 6-per-deoxy-6-per(2-carboxyethyl)thio-γ-cyclodextrin octasodium salt is isolated by precipitation.

Preferably said precipitation is obtained by cooling the reaction mixture, then adding water until completely dissolved, and finally adding an antisolvent selected from the group consisting of: methanol, and ethanol.

In this way, it is possible to obtain Sugammadex in solid form, which can then be recovered by filtration or any other solid-liquid separation technique known for this purpose to the skilled person in the art.

Although at the end of step c. obtained Sugammadex already has a high purity value, even higher than 99%, at the end of step c. of the process according to the present invention, Sugammadex can advantageously be further purified.

Preferably, the process according to the present invention comprises the step of:

d. purifying the 6-per-deoxy-6-per(2-carboxyethyl)thio-γ-cyclodextrin octasodium salt isolated in step c.

Preferably, said step d. comprises the steps of:

d-i. solubilizing the 6-per-deoxy-6-per(2-carboxyethyl)thio-γ-cyclodextrin octasodium salt isolated in step c. in an aqueous solvent, thus obtaining an aqueous solution;

d-ii. adding to the aqueous solution of step d-i. at least one activated carbon in an amount of from 0.01% to 25% by weight, preferably from 0.5% to 20% by weight, even more preferably from 1% to 10% by weight, with respect to the 6-per-deoxy-6-per(2-carboxyethyl)thio-γ-cyclodextrin octasodium salt, thus obtaining an aqueous suspension;

d-iii. filtering the aqueous suspension obtained in step d-ii., obtaining a filtrate comprising 6-per-deoxy-6-per(2-carboxyethyl)thio-γ-cyclodextrin octasodium salt; and

d-iv. separating from the filtrate of step d-iii. the 6-per-deoxy-6-per(2-carboxyethyl)thio-γ-cyclodextrin octasodium salt.

Preferably, in said step d-i. said aqueous solvent is water or a mixture comprising water and methanol, preferably in volume ratio water:methanol ranging from 1:3 to 4:1, more preferably from 1:1 to 4:1, and even more preferably from 2:1 to 4:1.

Preferably, in said step d-i. the 6-per-deoxy-6-per(2-carboxyethyl)thio-γ-cyclodextrin octasodium salt is present in said aqueous solution at a concentration ranging from 0.1 to 0.4 g/ml.

In said step d-ii. said activated carbon can be any activated carbon suitable for the purpose, such as for example activated carbon activated with zinc chloride or activated carbons marketed under the name ECOSORB (Graver Technologies LLC) and under the name Norit (Cabot Corporation).

Activated carbon activated with zinc chloride commercially available are for example those marketed under the name Wako (FUJIFILM Wako Pure Chemical Corporation) or Shirasagi A (Japan EnviroChemicals, Ltd.).

Preferably, in said step d-ii. said aqueous suspension is kept under stirring at a temperature from 10 to 40° C., for a time from 5 to 60 minutes.

Preferably, in said step d-iv. the 6-per-deoxy-6-per(2-carboxyethyl)thio-γ-cyclodextrin octasodium salt is separated from the filtrate by precipitation.

Preferably said precipitation is obtained by adding an antisolvent selected from the group consisting of: methanol, and ethanol. In this way, it is possible to obtain purified Sugammadex in solid form, which can then be recovered by filtration or any other solid-liquid separation technique known for this purpose to the person skilled in the art.

In a second aspect, the present invention also relates to a process for preparing a compound of formula (I)

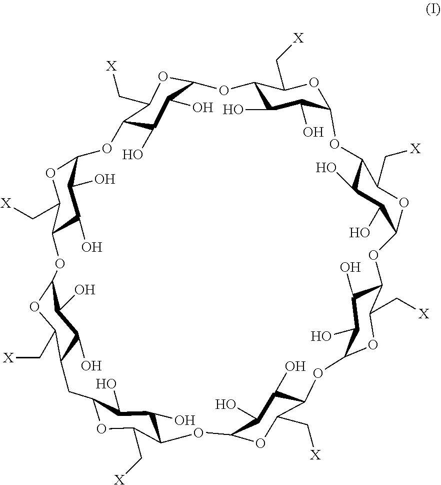

wherein X is selected from the group consisting of: CI, and Br, comprising the steps of:
A) reacting, in presence of at least one solvent selected from the group consisting of: toluene, and dichloromethane, at least one halide selected from the group consisting of: oxalyl halide, and thionyl halide, and a compound of formula (IV)

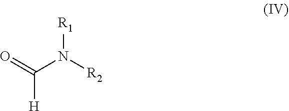

wherein R₁ and R₂ are —CH₃, phenyl groups or represent together a —CH₂—CH₂—O—CH₂—CH₂-group, thus obtaining a compound of formula (III)

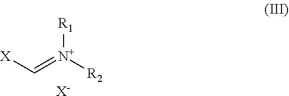

wherein
R₁, R₂ and X are as defined above;
B) distilling from the reaction mixture of step A) the at least one solvent selected from the group consisting of: toluene, and dichloromethane; and
C) reacting in presence of dimethylformamide at least one γ-cyclodextrin with the compound of formula (III) obtained from step B), thus obtaining the compound of formula (I).

Advantageously, said process for preparing the compound of formula (I) can be applied in any synthesis process of Sugammadex described in the prior art which provides for the involvement of the compound of formula (I) itself.

The Applicant has in fact found that the present process for preparing the compound of formula (I) allows obtaining high yields of the desired product under safe conditions and avoiding the formation of unwanted amounts of gaseous by-products which in their evolution can, on the one hand, give rise to phenomena of entrainment of the reaction product which limit its yield and, on the other hand, lead to accumulations in the equipment, for example in the condensers, reducing its efficiency.

The process for preparing the compound of formula (I) provides for a step A) reacting, in presence of at least one solvent selected from the group consisting of: toluene, and dichloromethane, at least one halide selected from the group consisting of: oxalyl halide , and thionyl halide, and a compound of formula (IV).

In step A) of the process for preparing the compound of formula (I) according to the present invention, the at least one halide and said compound of formula (IV) can be used in different reciprocal ratios.

In a preferred embodiment, in said step A) the at least one halide is used in an amount from 1.00 to 2.00 equivalents with respect to the equivalents of the compound of formula (IV), even more preferably in an amount from 1.05 to 1.9 equivalents with respect to the equivalents of the compound of formula (IV), even more preferably in an amount from 1.10 to 1.8 equivalents with respect to the equivalents of the compound of formula (IV). Preferably, in said embodiment, the excess halide is removed at the end of the reaction with the compound of formula (IV).

In a further particularly preferred embodiment, in said step A) the at least one halide is used in defect with respect to the equivalents of the compound of formula (IV), more preferably in an amount from 0.10 to 0.95 equivalents with respect to the equivalents of the compound of formula (IV), even more preferably in an amount from 0.40 to 0.80 equivalents with respect to the equivalents of the compound of formula (IV).

In step B) of the process for preparing the compound of formula (I), any distillation method suitable for the purpose for the person skilled in the art.

The process for preparing the compound of formula (I) provides for a step C) reacting in presence of dimethylformamide at least one γ-cyclodextrin with the compound of formula (III) obtained from step B), thus obtaining the compound of formula (I).

Preferably, said step C) is carried out at a temperature from 40 to 70° C.

Preferably, in said step C) the compound of formula (III) is used in an amount from 8 to 50 equivalents with respect to the equivalents of the at least one γ-cyclodextrin, more preferably from 12-40 equivalents with respect to the equivalents of the at least one γ-cyclodextrin, even more preferably from 20 to 35 equivalents with respect to the equivalents of the at least one γ-cyclodextrin.

Advantageously, the Applicant has further found that the process for preparing the compound of formula (I) according to the second aspect of the invention can be applied upstream of the process for preparing the 6-per-deoxy-6-per(2-carboxyethyl)thio-γ-cyclodextrin octasodium salt according to the first aspect of the present invention.

In a third aspect, the present invention therefore relates to a process for preparing the 6-per-deoxy-6-per(2-carboxyethyl)thio-γ-cyclodextrin octasodium salt, comprising the steps of:

(i) preparing a compound of formula (I) by means of the process according to the second aspect of the present invention;

(ii) preparing the 6-per-deoxy-6-per(2-carboxyethyl)thio-γ-cyclodextrin octasodium salt by means of the process according to the first aspect of the present invention.

Experimental Part

The invention is now described by means of some Examples to be considered for illustrative purposes and not as a limitation thereof.

### EXAMPLES

### Example 1

Oxalyl chloride (63.0 grams) was dripped into a reactor containing 120 milliliters of dimethylformamide (“DMF”) cooled to 0° C. and, at the end of the addition, the mixture was allowed to return to room temperature. During the reaction, the formation and evolution of gaseous by-products was observed, which gave rise to phenomena of entrainment of the reaction product and accumulations of product in the upper part of the reactor.

After having recovered the reaction product from the upper part of the reactor by using a spatula and added it to the remaining part of the reaction mixture, γ-cyclodextrin (20.0 grams) was thus added, then heating the reaction mixture thus obtained to 60° C. for 16 hours. Once complete conversion was observed, the reaction mixture was cooled to 25° C. and methanol (160 milliliters) was then added.

The solution thus obtained was dripped into a solution of water (360 milliliters) and methanol (200 milliliters) containing 63 grams of potassium bicarbonate kept at 25° C. In this way, the precipitation of 6-per-deoxy-6-per-chloro-γ-cyclodextrin was observed, which was then filtered under vacuum with a membrane pump and dried in an air-ventilated oven at 50° C. up to constant weight. 21 0 grams of product were thus obtained, with a reaction weight yield of 94.3%. The obtained product was also analysed by X-ray diffractometry using an X-ray diffractometer (operating with voltage of 45 kV, current of 40 mA, scanning speed of 0.025710 degrees per second, CuKα source, θ angle range from 3.0° to 49.992°).

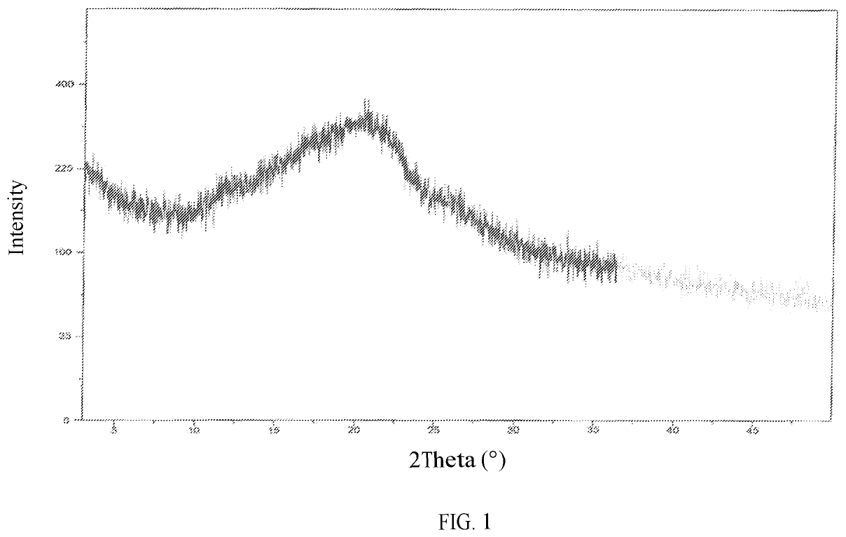

The X-ray analysis showed that the obtained product is amorphous (FIG. 1).

### Example 2

Oxalyl chloride (63.0 grams) was dripped into a reactor containing 200 milliliters of dichloromethane and 54.0 grams of dimethylformamide cooled to 0° C. and, at the end of the addition, the mixture was allowed to return to room temperature. During the reaction, the formation and evolution of gaseous by-products giving rise to phenomena of entrainment was not observed.

Further 100 milliliters of dimethylformamide were then added to the mixture thus obtained and dichloromethane was then removed by distillation. Subsequently, γ-cyclodextrin (20.0 grams) was then added to the mixture thus obtained, then heating the mixture to 60° C. for 16 hours. Once complete conversion was observed, the reaction mixture was cooled to 25° C. and methanol (160 milliliters) was then added.

The solution thus obtained was dripped into a solution of water (360 milliliters) and methanol (200 milliliters) containing 63 grams of potassium bicarbonate.

In this way, the precipitation of 6-per-deoxy-6-per-chloro-γ-cyclodextrin was observed, which was then filtered and dried like in Example 1, up to constant weight.

21.8 grams of product were thus obtained, with a reaction weight yield of 97.9%. The obtained product was also analysed by X-ray diffractometry, as described in Example 1.

The X-ray analysis showed that the obtained product is amorphous, with an X-ray spectrum similar to that obtained in Example 1.

### Example 3

Oxalyl chloride (48.5 grams) was dripped into a reactor containing 154 milliliters of toluene and 41.5 grams of dimethylformamide cooled to 0° C., and, at the end of the addition, the mixture was allowed to return to room temperature. During the reaction, the formation and evolution of gaseous by-products giving rise to phenomena of entrainment was not observed.

Further 77 milliliters of dimethylformamide were then added to the mixture thus obtained and toluene was then removed by distillation.

Subsequently, γ-cyclodextrin (15.3 grams) was then added to the mixture thus obtained, then heating the mixture to 60° C. for 16 hours. Once complete conversion was observed, the reaction mixture was cooled to 25° C. and methanol (123 milliliters) was then added.

The solution thus obtained was then dripped into a solution of water (277 milliliters) and methanol (154 milliliters) containing 48 grams of potassium bicarbonate.

In this way, the precipitation of 6-per-deoxy-6-per-chloro-γ-cyclodextrin was observed, which was then filtered and dried like in Example 1 up to constant weight.

15.8 grams of product were thus obtained, with a reaction weight yield of 93%. The obtained product was also analysed by X-ray diffractometry, as described in Example 1.

The X-ray analysis showed that the obtained product is amorphous, with an X-ray spectrum similar to that obtained in Example 1.

### Example 4

Oxalyl chloride (63.0 grams) was dripped into a reactor containing 200 milliliters of dichloromethane and 86.0 grams of N-formyl morpholine cooled to 0° C. and, at the end of the addition, the mixture was allowed to return to room temperature. During the reaction, the formation and evolution of gaseous by-products giving rise to phenomena of entrainment was not observed.

100 milliliters of dimethylformamide were then added to the mixture thus obtained and dichloromethane was then removed by distillation. Subsequently, γ-cyclodextrin (20.0 grams) was then added to the mixture thus obtained, then heating the mixture to 60° C. for 16 hours. Once complete conversion was observed, the reaction mixture was cooled to 25° C. and methanol (160 milliliters) was then added.

The solution thus obtained was dripped into a solution of water (360 milliliters) and methanol (200 milliliters) containing 63 grams of potassium bicarbonate.

In this way, the precipitation of 6-per-deoxy-6-per-chloro-γ-cyclodextrin was observed, which was then filtered and dried like in Example 1, up to constant weight.

20.6 grams of product were thus obtained, with a reaction weight yield of 92.5%. The obtained product was also analysed by X-ray diffractometry, as described in Example 1.

The X-ray analysis showed that the obtained product is amorphous, with an X-ray spectrum similar to that obtained in Example 1.

### Example 5

Oxalyl chloride (48.5 grams) was dripped into a reactor containing 154 milliliters of toluene and 48.0 grams of N-formyl morpholine cooled to 0° C. and, at the end of the addition, the mixture was allowed to return to room temperature. During the reaction, the formation and evolution of gaseous by-products giving rise to phenomena of entrainment was not observed.

77 milliliters of dimethylformamide were then added to the mixture thus obtained and toluene was then removed by distillation. Subsequently, γ-cyclodextrin (15.3 grams) was then added to the mixture thus obtained, then heating the mixture to 60° C. for 16 hours. Once complete conversion was observed, the reaction mixture was cooled to 25° C. and methanol (123 milliliters) was then added.

The solution thus obtained was dripped into a solution of water (277 milliliters) and methanol (154 milliliters) containing 48 grams of potassium bicarbonate.

In this way, the precipitation of 6-per-deoxy-6-per-chloro-γ-cyclodextrin was observed, which was then filtered and dried like in Example 1 up to constant weight.

16.6 grams of product were thus obtained, with a reaction weight yield of 97.4%. The obtained product was also analysed by X-ray diffractometry, as described in Example 1.

The X-ray analysis showed that the obtained product is amorphous, with an X-ray spectrum similar to that obtained in Example 1.

### Example 6

Oxalyl chloride (48.5 grams) was dripped into a reactor containing 154 milliliters of dichloromethane and 48.0 grams of N-formyl morpholine cooled to 0° C. and, at the end of the addition, the mixture was allowed to return to ambient temperatures. During the reaction, the formation and evolution of gaseous by-products giving rise to phenomena of entrainment was not observed.

The resulting suspension was filtered under vacuum with a membrane pump. The obtained solid was weighed recording a weight of 64.4 grams, indicating a reaction yield of 99%, and was then transferred to a new reactor, in which 100 milliliters of dimethylformamide and 15.3 grams of γ-cyclodextrin were added. The reaction mixture thus obtained was then heated to 60° C. for 16 hours. Once complete conversion was observed, the reaction mixture was cooled to 25° C. and methanol (123 milliliters) was then added.

The solution thus obtained was dripped into a solution of water (277 milliliters) and methanol (154 milliliters) containing 48 grams of potassium bicarbonate.

In this way, the precipitation of 6-per-deoxy-6-per-chloro-γ-cyclodextrin was observed, which was then filtered and dried like in Example 1 up to constant weight.

16.4 grams of product were thus obtained, with a reaction weight yield of 96.2%. The obtained product was also analysed by X-ray diffractometry, as described in Example 1.

The X-ray analysis showed that the obtained product is amorphous, with an X-ray spectrum similar to that obtained in Example 1.

### Example 7

The procedure of Example 6 was repeated, using toluene instead of dichloromethane. 16.7 grams of product were thus obtained, with a reaction weight yield of 98.0%. The obtained product was also analysed by X-ray diffractometry, as described in Example 1.

The X-ray analysis showed that the obtained product is amorphous, with an X-ray spectrum similar to that obtained in Example 1.

### Example 8

100 milliliters of dimethylformamide and 15.3 grams of γ-cyclodextrin were added into a reactor containing 48.3 g of N,N-dimethylchloromethylminium chloride (CAS Registry number: 3724-43-4). The reaction mixture thus obtained was then heated to 60° C. for 16 hours. Once complete conversion was observed, the reaction mixture was cooled to 25° C. and methanol (123 milliliters) was then added.

The solution thus obtained was dripped into a solution of water (277 milliliters) and methanol (154 milliliters) containing 48 grams of potassium bicarbonate.

In this way, the precipitation of 6-per-deoxy-6-per-chloro-γ-cyclodextrin was observed, which was then filtered and dried like in Example 1 up to constant weight.

16.8 grams of product were thus obtained, with a reaction weight yield of 97.6%. The obtained product was also analysed by X-ray diffractometry, as described in Example 1.

The X-ray analysis showed that the obtained product is amorphous, with an X-ray spectrum similar to that obtained in Example 1.

The Examples 2-8 have made it possible to appreciate how the process for preparing a compound of formula (I) according to the second aspect of the present invention allows reaching yield values that are always high, avoiding at the same time the formation of gaseous by-products which in their evolution give rise to phenomena of entrainment of the reaction product, thereby in addition not giving rise to undesired accumulations of product in the equipment used.

### Example 9

3-mercaptopropionic acid (1.9 grams) was added into a reactor containing 3.2 grams of sodium tert-butoxide and 32 milliliters of dimethylsulfoxide. The mixture thus obtained was stirred for 30 minutes at 25° C. After this time, 2 grams of amorphous 6-per-deoxy-6-per-chloro-gamma cyclodextrin obtained according to Example 2 were added, heating the reaction mixture to 60° C. During the reaction, the formation of a precipitate was observed. Representative samples of the reaction mixture were taken at regular intervals and analysed by high pressure liquid chromatography (HPLC) with the Waters Acquity-UV/DAD instrument set at a wavelength of 210 nm, to determine the amount of compound of formula (II) in the reaction mixture with respect to the total mass of reaction, by determining the percentage value of the chromatographic area of the compound of formula (II) with respect to the value of the chromatographic area of the total mass of reaction.

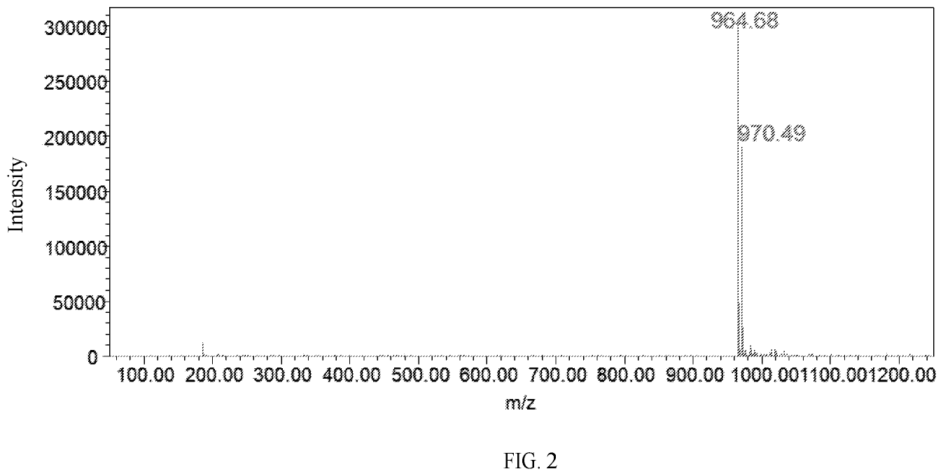

In the reaction mixture, the compound of formula (II) was identified by comparison of the retention times with a standard sample of the compound of formula (II) wherein X is CI, whose mass spectrum is reported by reference in FIG. 2, previously analysed by high pressure liquid chromatography (HPLC) with the same Waters Acquity-UV/DAD instrument set at the same wavelength of 210 nm.

When the amount of the compound of formula (II) in the reaction mixture, determined according to the foregoing, reached the value of 5% with respect to the total mass of reaction, 1 milliliter of water was added and the reaction was carried on until when the amount of the compound of formula (II) in the reaction mixture reached a value lower than 0.1% with respect to the total mass of reaction. The reaction mixture was then cooled to 25° C. and 40 milliliters of water were added. The complete dissolution of the precipitate was then observed and 120 milliliters of methanol were added, thus obtaining the precipitation of the reaction product, Sugammadex.

2.7 grams of Sugammadex were thus obtained, with a reaction weight yield of 90%.

### Comparative Example 1

1.9 grams of 3-mercaptopropionic acid and 1 milliliter of water were added into a reactor containing 3.2 grams of sodium tert-butoxide and 32 milliliters of dimethylsulfoxide. The mixture thus obtained was stirred for 30 minutes at 25° C. After this time, 2 grams of amorphous 6-per-deoxy-6-per-chloro-gamma cyclodextrin obtained according to Example 2 were added, heating the mixture to 60° C. During the reaction, the formation of a precipitate was observed. Representative samples of the reaction mixture were taken at regular intervals to determine the amount of the compound of formula (II) in the reaction mixture with respect to the total mass of reaction, as described in Example 9.

When the amount of the compound of formula (II) in the reaction mixture, determined according to the foregoing, reached a minimum constant value over time with respect to the total mass of reaction, the reaction mixture was cooled to 25° C. and 40 milliliters of water were added. The complete dissolution of the precipitate was then observed and 120 milliliters of methanol were added, thus obtaining the precipitation of the reaction product, Sugammadex.

2.5 grams of product were thus obtained, with a reaction weight yield of 83%.

### Comparative Example 2

1.9 grams of 3-mercaptopropionic acid were added into a reactor containing 3.2 grams of sodium tert-butoxide and 32 milliliters of dimethylsulfoxide. The mixture thus obtained was stirred for 30 minutes at 25° C. After this time, 2 grams of amorphous 6-per-deoxy-6-per-chloro-gamma cyclodextrin obtained according to Example 2 and 1 milliliter of water were added, heating the mixture to 60° C. During the reaction the formation of a precipitate was observed. Representative samples of the reaction mixture were taken at regular intervals to determine the amount of the compound of formula (II) in the reaction mixture with respect to the total mass of reaction, as described in Example 9.

When the amount of the compound of formula (II) in the reaction mixture, determined according to the foregoing, reached a minimum constant value over time with respect to the total mass of reaction, the reaction mixture was cooled to 25° C. and 40 milliliters of water were added. The complete dissolution of the precipitate was then observed and 120 milliliters of methanol were added, thus obtaining the precipitation of the reaction product, Sugammadex.

2.6 grams of product were thus obtained, with a reaction weight yield of 86%.

The comparison of Example 9 with Comparative Examples 1 and 2 made it possible to appreciate how the addition of a specific and defined amount of water in the reaction mixture in a specific stage of progress of said substitution reaction, allows to improve the reaction yield in an evident way, which in Example 9 is equal to 90% and in the comparative examples 1 and 2, in which water was added before and at the time of adding the compound (I), respectively, equal to values of 83 and 86%, respectively. It has also been possible to appreciate that the higher reaction yield has also ensured at the end of the reaction the presence of lesser amounts of partial substitution products that are difficult to separate from the product, thereby making the need to resort, for subsequent purifications, to complex and expensive purification techniques such as chromatographic methods, molecular exclusion membranes and ion exchange resins completely superfluous.

### Example 10

3-mercaptopropionic acid (2.9 grams) was added into a reactor containing 4.8 grams of sodium tert-butoxide and a binary mixture of 30 milliliters of dimethylsulfoxide and 18 milliliters of tetrahydrofuran. The mixture thus obtained was stirred for 30 minutes at 25° C. After this time, 3 grams of amorphous 6-per-deoxy-6-per-chloro-gamma cyclodextrin obtained according to Example 2 were added, heating the mixture to 60° C. During the reaction, the formation of a precipitate was observed. Representative samples of the reaction mixture were taken at regular intervals to determine the amount of the compound of formula (II) in the reaction mixture with respect to the total mass of reaction, as described in Example 9.

When the amount of the compound of formula (II) in the reaction mixture, determined according to the foregoing, reached the value of 5% with respect to the total mass of reaction, 1.5 milliliters of water were added and the reaction was carried on until when the amount of the compound of formula (II) in the reaction mixture reached the value of 0.1% with respect to the total mass of reaction. The reaction mixture was then cooled to 25° C. and 60 milliliters of water were added.

The complete dissolution of the precipitate was then observed and 180 milliliters of methanol were added, thus obtaining the precipitation of the reaction product, Sugammadex.

4 grams of product were thus obtained, with a reaction weight yield of 88%.

### Example 11

3-mercaptopropionic acid (4.4 grams) was added into a reactor containing 7.2 grams of sodium tert-butoxide and a binary mixture of 45 milliliters of dimethylsulfoxide and 27 milliliters of N,N-dimethylformamide. The mixture thus obtained was stirred for 30 minutes at 25° C. After this time, 4.5 grams of amorphous 6-per-deoxy-6-per-chloro-gamma cyclodextrin obtained according to Example 2 were added, heating the mixture to 60° C. During the reaction, the formation of a precipitate was observed. Representative samples of the reaction mixture were taken at regular intervals to determine the amount of the compound of formula (II) in the reaction mixture with respect to the total mass of reaction, as described in Example 9.

When the amount of the compound of formula (II) in the reaction mixture, determined according to the foregoing, reached the value of 5% with respect to the total mass of reaction, 2.3 milliliters of water were added and the reaction was carried on until when the amount of the compound of formula (II) in the reaction mixture reached the value of 0.1% with respect to the total mass of reaction. The reaction mixture was then cooled to 25° C. and 90 milliliters of water were added.

The complete dissolution of the precipitate was then observed and 270 milliliters of methanol were added, thus obtaining the precipitation of the reaction product, Sugammadex.

5.8 grams of product were thus obtained, with a reaction weight yield of 85%.

### Example 12

3-mercaptopropionic acid (2.9 grams) was added into a reactor containing 4.8 grams of sodium tert-butoxide and a binary mixture of 30 milliliters of dimethylsulfoxide and 18 milliliters of N,N-dimethylacetamide. The mixture thus obtained was stirred for 30 minutes at 25° C. After this time, 3 grams of amorphous 6-per-deoxy-6-per-chloro-gamma cyclodextrin obtained according to Example 2 were added, heating the mixture to 60° C. During the reaction, the formation of a precipitate was observed. Representative samples of the reaction mixture were taken at regular intervals to determine the amount of the compound of formula (II) in the reaction mixture with respect to the total mass of reaction, as described in Example 9.

When the amount of the compound of formula (II) in the reaction mixture, determined according to the foregoing, reached the value of 5% with respect to the total mass of reaction, 1.5 milliliters of water were added and the reaction was carried on until when the amount of the compound of formula (II) in the reaction mixture reached the value of 0.1% with respect to the total mass of reaction. The reaction mixture was then cooled to 25° C. and 60 milliliters of water were added.

The complete dissolution of the precipitate was then observed and 180 milliliters of methanol were added, thus obtaining the precipitation of the reaction product, Sugammadex.

3.7 grams of product were thus obtained, with a reaction weight yield of 81%.

### Example 13

3-mercaptopropionic acid (5.8 grams) was added into a reactor containing 9.6 grams of sodium tert-butoxide and a binary mixture of 60 milliliters of dimethylsulfoxide and 36 milliliters of N-methylpyrrolidone. The mixture thus obtained was stirred for 30 minutes at 25° C. After this time, 6 grams of amorphous 6-per-deoxy-6-per-chloro-gamma cyclodextrin obtained according to Example 2 were added, heating the mixture to 60° C. During the reaction, the formation of a precipitate was observed. Representative samples of the reaction mixture were taken at regular intervals to determine the amount of the compound of formula (II) in the reaction mixture with respect to the total mass of reaction, as described in Example 9.

When the amount of the compound of formula (II) in the reaction mixture, determined according to the foregoing, reached the value of 5% with respect to the total mass of reaction, 1.5 milliliters of water were added and the reaction was carried on until when the amount of the compound of formula (II) in the reaction mixture reached the value of 0.1% with respect to the total mass of reaction. The reaction mixture was then cooled to 25° C. and 120 milliliters of water were added.

The complete dissolution of the precipitate was then observed and 360 milliliters of methanol were added, thus obtaining the precipitation of the reaction product, Sugammadex.

7.8 grams of product were thus obtained, with a reaction weight yield of 86%.

### Example 14

4.8 grams of sodium tert-butoxide dissolved in 18 milliliters of tetrahydrofuran and 2.9 grams of 3-mercaptopropionic acid were added into a reactor containing a mixture of 30 milliliters of dimethylsulfoxide and 18 milliliters of N-methylpyrrolidone. The mixture thus obtained was stirred for 30 minutes at 25° C. After this time, 3 grams of amorphous 6-per-deoxy-6-per-chloro-gamma cyclodextrin obtained according to Example 2 were added, heating the mixture to 60° C. During the reaction, the formation of a precipitate was observed. Representative samples of the reaction mixture were taken at regular intervals to determine the amount of the compound of formula (II) in the reaction mixture with respect to the total mass of reaction, as described in Example 9.

When the amount of the compound of formula (II) in the reaction mixture, determined according to the foregoing, reached the value of 5% with respect to the total mass of reaction, 1.5 milliliters of water were added and the reaction was carried on until when the amount of the compound of formula (II) in the reaction mixture reached the value of 0.1% with respect to the total mass of reaction. The reaction mixture was then cooled to 25° C. and 60 milliliters of water were added.

The complete dissolution of the precipitate was then observed and 180 milliliters of methanol were added, thus obtaining the precipitation of the reaction product, Sugammadex.

4 grams of product were thus obtained, with a reaction weight yield of 88%.

### Example 15

1.9 grams of 3-mercaptopropionic acid were added into a reactor containing 1.8 grams of sodium methoxide and 32 milliliters of dimethylsulfoxide. The mixture thus obtained was stirred for 30 minutes at 25° C. After this time, 2 grams of amorphous 6-per-deoxy-6-per-chlorine-gamma cyclodextrin obtained from Example 2 were added, heating the mixture to 60° C. During the reaction, the formation of a precipitate was observed. Representative samples of the reaction mixture were taken at regular intervals to determine the amount of the compound of formula (II) in the reaction mixture with respect to the total mass of reaction, as described in Example 9.

When the amount of the compound of formula (II) in the reaction mixture, determined according to the foregoing, reached the value of 5% with respect to the total mass of reaction, 1 milliliter of water was added and the reaction was carried on until when the amount of the compound of formula (II) in the reaction mixture reached the value of 0.1% with respect to the total mass of reaction. The reaction mixture was then cooled to 25° C. and 40 milliliters of water were added.

The complete dissolution of the precipitate was then observed and 120 milliliters of methanol were added, thus obtaining the precipitation of the reaction product, Sugammadex.

2.7 grams of product were thus obtained, with a reaction weight yield of 90%.

### Example 16

The procedure of Example 15 was repeated, using 2.2 grams of sodium ethoxide instead of 1.8 grams of sodium methoxide, obtaining, also in this case, 2.7 grams of product, with a reaction weight yield of 90%.

### Example 17

The procedure of Example 15 was repeated, using 3.6 grams of sodium tert-pentoxide instead of 1.8 grams of sodium methoxide, obtaining, also in this case, 2.7 grams of product, with a reaction weight yield of 90%.

### Example 18

1.9 grams of 3-mercaptopropionic acid were added into a reactor containing 3.2 grams of sodium tert-butoxide and a mixture of 32 milliliters of dimethylsulfoxide and 1.5 milliliters of methanol. The mixture thus obtained was stirred for 30 minutes at 25° C. After this time, 2 grams of amorphous 6-per-deoxy-6-per-chlorine-gamma cyclodextrin obtained from Example 2 were added, heating the mixture to 60° C. During the reaction, the formation of a precipitate was observed. Representative samples of the reaction mixture were taken at regular intervals to determine the amount of the compound of formula (II) in the reaction mixture with respect to the total mass of reaction, as described in Example 9.

When the amount of the compound of formula (II) in the reaction mixture, determined according to the foregoing, reached the value of 5% with respect to the total mass of reaction, 1 milliliter of water was added and the reaction was carried on until when the amount of the compound of formula (II) in the reaction mixture reached the value of 0.1% with respect to the total mass of reaction. The reaction mixture was then cooled to 25° C. and 40 milliliters of water were added.

The complete dissolution of the precipitate was then observed and 120 milliliters of methanol were added, thus obtaining the precipitation of the reaction product, Sugammadex.

2.6 grams of product were thus obtained, with a reaction weight yield of 86%.

### Example 19

The procedure of Example 18 was repeated, using a mixture of 32 milliliters of dimethylsulfoxide and 2.2 milliliters of ethanol instead of the mixture of 32 milliliters of dimethylsulfoxide and 1.5 milliliters of methanol.

2.5 grams of product were thus obtained, with a reaction weight yield of 83%.

### Example 20

The procedure of Example 18 was repeated, using a mixture of 32 milliliters of dimethylsulfoxide and 2.7 milliliters of n-propanol instead of the mixture of 32 milliliters of dimethylsulfoxide and 1.5 milliliters of methanol.

2.9 grams of product were thus obtained, with a reaction weight yield of 97%.

### Example 21

The procedure of Example 18 was repeated, using a mixture of 32 milliliters of dimethylsulfoxide and 2.8 milliliters of isopropanol instead of the mixture of 32 milliliters of dimethylsulfoxide and 1.5 milliliters of methanol.

2.7 grams of product were thus obtained, with a reaction weight yield of 90%.

### Example 22

The procedure of Example 18 was repeated, using a mixture of 32 milliliters of dimethylsulfoxide and 3.3 milliliters of n-butanol instead of the mixture of 32 milliliters of dimethylsulfoxide and 1.5 milliliters of methanol.

2.4 grams of product were thus obtained, with a reaction weight yield of 80%.

### Example 23

The procedure of Example 18 was repeated, using a mixture of 32 milliliters of dimethylsulfoxide and 3.4 milliliters of iso-butanol instead of the mixture of 32 milliliters of dimethylsulfoxide and 1.5 milliliters of methanol. 2.7 grams of product were thus obtained, with a reaction weight yield of 90%.

### Example 24

The procedure of Example 18 was repeated, using a mixture of 32 milliliters of dimethylsulfoxide and 2.1 milliliters of ethylene glycol instead of the mixture of 32 milliliters of dimethylsulfoxide and 1.5 milliliters of methanol.

2.7 grams of product were thus obtained, with a reaction weight yield of 90%.

### Example 25

3-mercaptopropionic acid (37 grams) was added into a reactor containing 63.4 grams of sodium tert-butoxide and 640 milliliters of dimethylsulfoxide. The mixture thus obtained was stirred for 30 minutes at 25° C. After this time, 40 grams of amorphous 6-per-deoxy-6-per-chloro-gamma cyclodextrin obtained according to Example 2 were added, heating the reaction mixture to 60° C. During the reaction, the formation of a precipitate was observed. Representative samples of the reaction mixture were taken at regular intervals to determine the amount of the compound of formula (II) in the reaction mixture with respect to the total mass of reaction, as described in Example 9.

When the amount of the compound of formula (II) in the reaction mixture, determined according to the foregoing, reached the value of 5% with respect to the total mass of reaction, 20 milliliters of water were added and the reaction was carried on until when the amount of the compound of formula (II) in the reaction mixture reached the value of 0.1% with respect to the total mass of reaction. The reaction mixture was then cooled to 25° C. and 800 milliliters of water were added. The complete dissolution of the precipitate was then observed and 2400 milliliters of methanol were added, thus obtaining the precipitation of the reaction product, Sugammadex.

56 grams of product were thus obtained, with a reaction weight yield of 93%

### Example 26

An aqueous solution of Sugammadex was prepared by dissolving 5 grams of the product obtained according to Example 25 into 20 milliliters of water. 0.5 grams of special grade WAKO activated carbon (FUJIFILM Wako Pure Chemical Corporation) were then added to the aqueous solution and the suspension thus obtained was stirred for 30 minutes at 25° C. After removal of the activated carbon by filtration on paper, 40 milliliters of methanol were added to the filtrate, observing the precipitation of the purified Sugammadex.

4.9 grams of purified Sugammadex were thus obtained, with a purification weight yield of 98%.

In accordance with the guidelines “Impurities in new drug substances” (Q3A) of the International Council for Harmonisation of Technical Requirements for Pharmaceuticals for Human Use (ICH), purified Sugammadex showed an HPLC purity greater than 99.5%, a content of unknown impurities lower than 0.10%, and a content of known impurities lower than 0.1% (amount of compound of formula (II) <0.05%).

### Example 27

An aqueous solution of Sugammadex was prepared by dissolving 7 grams of the product obtained according to Example 25 into 28 milliliters of water. 0.7 grams of special grade WAKO activated carbon were then added to the aqueous solution and the suspension thus obtained was stirred for 30 minutes at 25° C. After removal of the activated carbon by filtration on paper, 100 milliliters of ethanol were added to the filtrate, observing the precipitation of the purified Sugammadex.

6.8 grams of purified Sugammadex were thus obtained, with a purification weight yield of 97%.

The obtained Sugammadex showed a profile of impurities quite similar to that of Sugammadex according to Example 26.

Example 28

An aqueous solution of Sugammadex was prepared by dissolving 3 grams of the product obtained according to Example 25 into a mixture consisting of 9 milliliters of water and 3 milliliters of methanol. 0.3 grams of special grade WAKO activated carbon were then added to the aqueous solution and the suspension thus obtained was stirred for 30 minutes at 25° C. After removal of the activated carbon by filtration on paper, 50 milliliters of methanol were added to the filtrate, observing the precipitation of the purified Sugammadex.

2.8 grams of purified Sugammadex were thus obtained, with a purification weight yield of 93%.

The obtained Sugammadex showed a profile of impurities quite similar to that of Sugammadex according to Example 26.

### Example 29

An aqueous solution of Sugammadex was prepared by dissolving 5 grams of the product obtained according to Example 25 into 20 milliliters of water. 0.7 grams of SHIRASAGI A (Japan EnviroChemicals, Ltd.) activated carbon were then added to the aqueous solution and the suspension thus obtained was stirred for 30 minutes at 25° C. After removal of the activated carbon by filtration on paper, 40 milliliters of ethanol were added to the filtrate, observing the precipitation of the purified Sugammadex.

4.9 grams of purified Sugammadex were thus obtained, with a purification weight yield of 98%.

The obtained Sugammadex showed a profile of impurities quite similar to that of Sugammadex according to Example 26.

### Example 30

An aqueous solution of Sugammadex was prepared by dissolving 7 grams of the product obtained according to Example 25 into 28 milliliters of water. 0.7 grams of SHIRASAGI A activated carbon were then added to the aqueous solution and the suspension thus obtained was stirred for 30 minutes at 25° C. After removal of the activated carbon by filtration on paper, 100 milliliters of ethanol were added to the filtrate, observing the precipitation of the purified Sugammadex.

6.8 grams of purified Sugammadex were thus obtained, with a purification weight yield of 97%.

The obtained Sugammadex showed a profile of impurities quite similar to that of Sugammadex according to Example 26.

Example 31

An aqueous solution of Sugammadex was prepared by dissolving 3 grams of the product obtained according to Example 25 into a mixture consisting of 9 milliliters of water and 3 milliliters of methanol. 0.3 grams of SHIRASAGI A activated carbon were then added to the aqueous solution and the suspension thus obtained was stirred for 30 minutes at 25° C. After removal of the activated carbon by filtration on paper, 50 milliliters of ethanol were added to the filtrate, observing the precipitation of the purified Sugammadex.

2.8 grams of purified Sugammadex were thus obtained, with a purification weight yield of 93%.

The obtained Sugammadex showed a profile of impurities quite similar to that of Sugammadex according to Example 26.

Example 32

An aqueous solution of Sugammadex was prepared by dissolving 5 grams of the product obtained according to Example 25 into 20 milliliters of water. 0.25 grams of ECOSORB C-948 (Graver Technologies LLC) activated carbon were then added to the aqueous solution and the suspension thus obtained was stirred for 30 minutes at 25° C. After removal of the activated carbon by filtration on paper, 40 milliliters of methanol were added to the filtrate, observing the precipitation of the purified Sugammadex.

4.9 grams of purified Sugammadex were thus obtained, with a purification weight yield of 98%.

The obtained Sugammadex showed a profile of impurities quite similar to that of Sugammadex according to Example 26.

### Example 33

An aqueous solution of Sugammadex was prepared by dissolving 5 grams of the product obtained according to Example 25 into 20 milliliters of water. 0.25 grams of ECOSORB C-906 (Graver Technologies LLC) activated carbon were then added to the aqueous solution and the suspension thus obtained was stirred for 30 minutes at 25° C.

After removal of the activated carbon by filtration on paper, 40 milliliters of methanol were added to the filtrate, observing the precipitation of the purified Sugammadex.

4.9 grams of purified Sugammadex were thus obtained, with a purification weight yield of 98%.

The obtained Sugammadex showed a profile of impurities quite similar to that of Sugammadex according to Example 26.

### Example 34

An aqueous solution of Sugammadex was prepared by dissolving 5 grams of the product obtained according to Example 25 into 20 milliliters of water. 0.25 grams of NORIT SX ULTRA (Cabot Corporation) activated carbon were then added to the aqueous solution and the suspension thus obtained was stirred for 30 minutes at 25° C. After removal of the activated carbon by filtration on paper, 40 milliliters of methanol were added to the filtrate, observing the precipitation of the purified Sugammadex.

4.8 grams of purified Sugammadex were thus obtained, with a purification yield of 96%.

The obtained Sugammadex showed a profile of impurities quite similar to that of Sugammadex according to Example 26.

### Example 35

An aqueous solution of Sugammadex was prepared by dissolving 5 grams of the product obtained according to Example 25 into 20 milliliters of water. 0.25 grams of NORIT SX PLUS activated carbon were then added to the aqueous solution and the suspension thus obtained was stirred for 30 minutes at 25° C. After removal of the activated carbon by filtration on paper, 40 milliliters of methanol were added to the filtrate, observing the precipitation of the purified Sugammadex.

4.7 grams of purified Sugammadex were thus obtained, with a purification weight yield of 94%.

The obtained Sugammadex showed a profile of impurities quite similar to that of Sugammadex according to Example 26.

The analysis of the results of the Examples 26-35 made it possible to appreciate how the process according to the present invention allows obtaining high yields of Sugammadex with a degree of purity in accordance with the guidelines “Impurities in new drug substances” (Q3A) of the ICH, without the need to resort to the use of complex and expensive purification techniques such as chromatographic methods, thereby contributing to the competitiveness of the process itself.

### Example 36

Oxalyl chloride (63.0 grams) was dripped into a reactor containing 200 milliliters of dichloromethane and 54.0 grams of dimethylformamide cooled to 0° C. and, at the end of the addition, the mixture was allowed to return to room temperature. During the reaction, the formation and evolution of gaseous by-products giving rise to phenomena of entrainment was not observed.

Further 100 milliliters of dimethylformamide were then added to the mixture thus obtained and dichloromethane was then removed by distillation. Subsequently, γ-cyclodextrin (20.0 grams) was then added to the mixture thus obtained, then heating the mixture to 40° C. for 16 hours. Once complete conversion was observed, the reaction mixture was cooled to 25° C. and methanol (160 milliliters) was then added.

The solution thus obtained was dripped into a solution of water (360 milliliters) and methanol (200 milliliters) containing 63 grams of potassium bicarbonate.

In this way, the precipitation of 6-per-deoxy-6-per-chloro-γ-cyclodextrin was observed, which was then filtered and dried like in Example 1, up to constant weight.

21.0 grams of product were thus obtained, with a reaction weight yield of 94.3%. The obtained product was also analysed by X-ray diffractometry, as described in Example 1.

The X-ray analysis showed that the obtained product is amorphous, with an X-ray spectrum similar to that obtained in Example 1.

### Example 37

Oxalyl chloride (63.0 grams) was dripped into a reactor containing 200 milliliters of dichloromethane and 54.0 grams of dimethylformamide cooled to 0° C. and, at the end of the addition, the mixture was allowed to return to room temperature. During the reaction, the formation and evolution of gaseous by-products giving rise to phenomena of entrainment was not observed.

Further 100 milliliters of dimethylformamide were then added to the mixture thus obtained and dichloromethane was then removed by distillation. Subsequently, γ-cyclodextrin (20.0 grams) was then added to the mixture thus obtained, then heating the mixture to 50 ° C. for 16 hours. Once complete conversion was observed, the reaction mixture was cooled to 25° C. and methanol (160 milliliters) was then added.

The solution thus obtained was dripped into a solution of water (360 milliliters) and methanol (200 milliliters) containing 63 grams of potassium bicarbonate.

In this way, the precipitation of 6-per-deoxy-6-per-chloro-γ-cyclodextrin was observed, which was then filtered and dried like in Example 1, up to constant weight.

21.4 grams of product were thus obtained, with a reaction weight yield of 96.1%. The obtained product was also analysed by X-ray diffractometry, as described in Example 1.

The X-ray analysis showed that the obtained product is amorphous, with an X-ray spectrum similar to that obtained in Example 1.

1. A process for preparing 6-per-deoxy-6-per(2-carboxyethyl)thio-γ-cyclodextrin octasodium salt, comprising the steps of:
a. reacting a compound of formula (I) 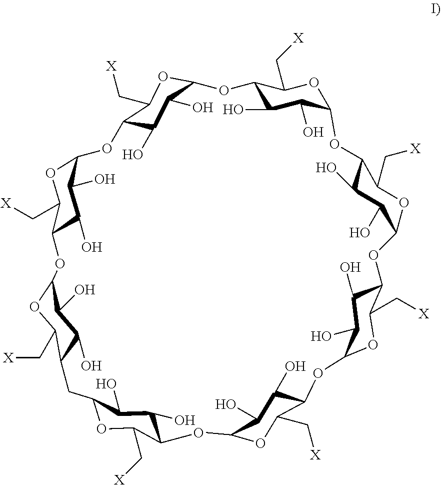
wherein X is selected from the group consisting of: Cl, and Br, with 3-mercaptopropionic acid in presence of at least one sodium alkoxide and of at least one aprotic organic solvent;
b. adding to the reaction mixture of step a. water, in an amount of from 0.5% to 10% by volume with respect to the total volume of said at least one aprotic organic solvent, when the compound of formula (II) 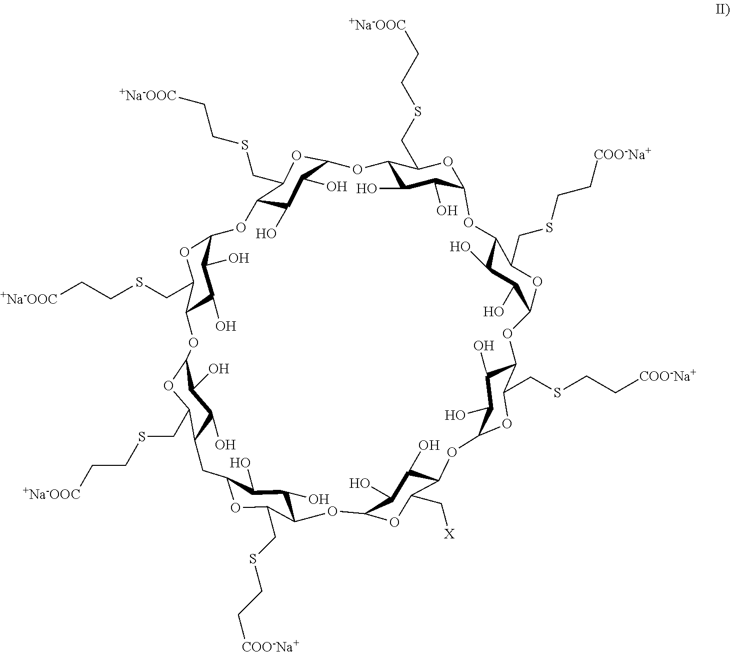
wherein X is as defined above, is present in the reaction mixture in an amount equal to or lower than 10% with respect to the total mass of reaction; and
c. isolating the 6-per-deoxy-6-per(2-carboxyethyl)thio-γ-cyclodextrin octasodium salt from the total mass of reaction obtained from step b.

2. The process according to claim 1, wherein said compound of formula (I) is prepared before or during said step a., by means of a process comprising reacting at least one γ-cyclodextrin with at least one halogenating agent in presence of dimethylformamide.

3. The process according to claim 2, wherein the at least one halogenating agent is a compound of formula (III) 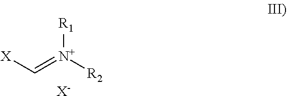
wherein
R₁ and R₂ are —CH₃, phenyl groups or represent together a-CH₂—CH₂—O—CH₂—CH₂-group; and
X is selected from the group consisting of: Cl, and Br.

4. The process according to claim 3, wherein said compound of formula (III) is obtained before reacting the at least one γ-cyclodextrin with the at least one halogenating agent in presence of dimethylformamide by means of the steps of:
reacting, in presence of at least one solvent selected from the group consisting of: toluene, and dichloromethane, at least one halide selected from the group consisting of: oxalyl halide, and thionyl halide, and a compound of formula (IV) 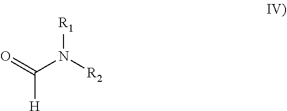
wherein R₁ and R₂ are —CH₃, phenyl groups or represent together a —CH₂—CH₂—O—CH₂—CH₂— group; and
distilling the at least one solvent selected from the group consisting of: toluene, and dichloromethane.

5. The process according to claim 1, wherein in said step a. the at least one sodium alkoxide is selected from the group consisting of sodium tert-butoxide, sodium methoxide, sodium ethoxide, sodium tert-pentoxide.

6. The process according to claim 1, wherein in said step a. the at least one aprotic organic solvent is dimethylsulfoxide or a mixture of solvents comprising dimethylsulfoxide and at least one other solvent selected from the group consisting of: tetrahydrofuran, dimethylformamide, dimethylacetamide, N-methylpyrrolidone, methanol, ethanol, n-propanol, isopropanol, n-butanol, isobutanol, and ethylene glycol.

7. The process according to claim 1, wherein in said step b. the amount of compound of formula (II) in the total mass of reaction is measured by means of high pressure liquid chromatography (HPLC), determining the percentage value of the chromatographic area of the compound of formula (II) with respect to the value of the chromatographic area of the total reaction mass.

8. The process according to claim 1, comprising the steps of:
d. purifying the 6-per-deoxy-6-per(2-carboxyethyl)thio-γ-cyclodextrin octasodium salt isolated in step c.

9. The process according to claim 8, wherein said step d. comprises the steps of:
d-i. solubilizing the 6-per-deoxy-6-per(2-carboxyethyl)thio-γ-cyclodextrin octasodium salt isolated in step c. in an aqueous solvent, thus obtaining an aqueous solution;
d-ii. adding to the aqueous solution of step d-i. at least one activated carbon in an amount of from 0.01% to 25% by weight with respect to the 6-per-deoxy-6-per(2-carboxyethyl)thio-γ-cyclodextrin octasodium salt, thus obtaining an aqueous suspension;
d-iii. filtering the aqueous suspension obtained in step d-ii., obtaining a filtrate comprising 6-per-deoxy-6-per(2-carboxyethyl)thio-γ-cyclodextrin octasodium salt; and
d-iv. separating from the filtrate of step d-iii. the 6-per-deoxy-6-per(2-carboxyethyl)thio-γ-cyclodextrin octasodium salt.

10. A process for preparing a compound of formula (I) 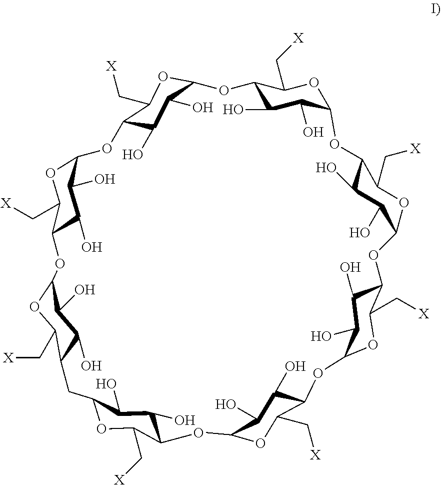
wherein X is selected from the group consisting of: Cl, and Br, comprising the steps of:
A) reacting, in presence of at least one solvent selected from the group consisting of: toluene, and dichloromethane, at least one halide selected from the group consisting of: oxalyl halide, and thionyl halide, and a compound of formula (IV) 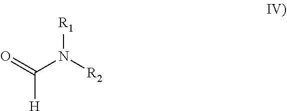
wherein R₁ and R₂ are —CH₃, phenyl groups or represent together a —CH₂—CH₂—O—CH₂-CH₂-group, thus obtaining a compound of formula (III) 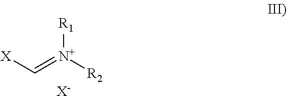
wherein
R₁, R₂ and X are as defined above;
B) distilling from the reaction mixture of step A) the at least one solvent selected from the group consisting of: toluene, and dichloromethane; and
C) reacting in presence of dimethylformamide at least one γ-cyclodextrin with the compound of formula (III) obtained from step B), thus obtaining the compound of formula (I).

11. A process for preparing the 6-per-deoxy-6-per(2-carboxyethyl)thio-γ-cyclodextrin octasodium salt, comprising the steps of:
(i) preparing a compound of formula (I) by means of the process according to claim 10;
(ii) preparing the 6-per-deoxy-6-per(2-carboxyethyl)thio-γ-cyclodextrin octasodium salt by means of the process according to claim 1.
